(19) United States
(12) Patent Application Publication
(10) Pub. No.: US 2024/0270669 A1
(43) Pub. Date: Aug. 15, 2024

(54) METHOD FOR PREPARING 2-IODO ARYL ETHER UNDER THE ACTION OF ALKALI METAL HYDRIDE

(71) Applicant: SOOCHOW UNIVERSITY, Suzhou (CN)

(72) Inventors: Shilei ZHANG, Suzhou (CN); Wenjing ZHU, Suzhou (CN); Chunhui FANG, Suzhou (CN); Yanwei HU, Suzhou (CN); Xin CHEN, Suzhou (CN); Jiawen HUANG, Suzhou (CN)

(21) Appl. No.: 18/037,553

(22) PCT Filed: Feb. 23, 2021
(86) PCT No.: PCT/CN2021/077530

§ 371 Date: Oct. 31, 2023

(51) Int. Cl. C07C 41/16; C07C 41/34

(52) U.S. Cl. CPC C07C 41/16; C07C 41/34

(57) ABSTRACT

Disclosed in the present invention is a method for preparing 2-iodo aryl ether under the action of alkali metal hydride: adding alkali metal hydride and phenol to a solvent, then adding 1,2-diiodoarene, and reacting at 0-100° C. to obtain a 2-iodo aryl ether product. The coupling process of the present invention does not require the addition of a transition metal catalyst, and does not cause metal contamination to the product; the method of the present invention can be performed at room temperature and has high functional group compatibility, and solves the problem that existing metal-catalysed coupling to aryl ether reactions need to be performed at a relatively high temperature.

### TECHNICAL FIELD

The present invention belongs to the technical field of organic synthesis, and particularly relates to a method for preparing 2-iodo-aromatic ether by a coupling reaction of an aryne intermediate generated by ortho-iodine halide under the action of alkali metal hydride and phenol.

### BACKGROUND OF INVENTION

2-Iodo-aromatic ethers are very useful organic building blocks that can be used for the synthesis of various oxygen-containing heterocycles, as well as for the diverse conversion of iodine to prepare ortho-substituted aromatic ether derivatives. In the prior art, the formation of carbon-carbon or carbon-heteroatomic bond requires metal catalysts or strong bases as promoters; alternatively, the complex precursors can be used, for example, the compound 1 is used as the precursor to react for 12 hours in the presence of cesium carbonate to obtain iodo-aromatic ethers.

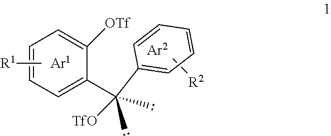

The existing technologies use the noble metal catalysts to catalyze the mono-halogen to substitute the aromatic hydrocarbon for the reaction with phenol, but there have been no reports of the reaction using dihalogen substituted aromatic hydrocarbon with phenol.

### Technical Problems

The present invention provides a method for synthesizing 2-iodo-aromatic ether in one step at room temperature; the ortho-iodine halide is used for the first time to absorb halogen atoms of another molecule of ortho-iodine halide under the action of alkali metal hydride to form a 2-iodo-aromatic ether product. The present invention is a coupling reaction of phenol promoted by alkali metal hydrides and ortho-iodo halides. This type of conversion is fast, simple, and mild, and it does not require the catalysis of transition metal, and has no by-products of over-coupling. It is a novel method to quickly construct various 2-iodo-aromatic ethers.

### Technical Solution

The technical means of synthesizing the 2-iodo-aromatic ether is to mix alkali metal hydride, phenol and 1, 2-iodo-aromatic hydrocarbon in a solvent and stirred to obtain the product without other reagents. The specific technical scheme is as follows: a method for preparing 2-iodo-aromatic ether under the action of alkali metal hydrides comprises the following steps: 1, 2-iodo-aromatic hydrocarbon reacts with phenol in the presence of alkali metal hydride to obtain 2-iodo-aromatic ether; preferably, adding alkali metal hydride and phenol into a solvent, then adding 1, 2-iodo-aromatic hydrocarbon to react at 0-100° C. to obtain the product of 2-iodo-aromatic ether.

In the present invention, the phenol is phenol, substituted phenol or heterocyclic phenol.

In the present invention, the alkali metal hydride is sodium hydride, potassium hydride and lithium hydride, preferably sodium hydride and potassium hydride, more preferably sodium hydride.

In the present invention, the solvent is one or several of DMF (N,N-dimethylformamide), DMA (N,N-Dimethylacetamide), THF (tetrahydrofuran), 2-MeTHF (2-methyltetrahydrofuran), DME (ethylene glycol dimethyl ether), MTBE (methyl tert-butyl ether), diethyl ether, DMSO, NMP (N-methyl-2-pyrrolidone), toluene, preferably DMA and THF, more preferably a mixture of DMA and THF.

In the present invention, the mole ratio of the phenol to the 1,2-iodo-aromatic hydrocarbon to the alkali metal hydride is 1:(0.1-10):(1.2-10); preferably 1:(0.5-5):(1.5-5); more preferably 1:(1-3):(2-4); most preferably 1:1.5:3.

In the technical scheme, the reaction temperature is 0-100° C., preferably 10-50° C., more preferably the room temperature; the reaction time is 0.2-10 hours, preferably 0.5-2 hours.

In the present invention, the chemical structural formula of 1, 2-iodo-aromatic hydrocarbon is as follows:

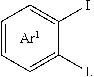

In the present invention, the chemical structural formula of phenol is Ar²OH.

In the present invention, the chemical structural formula of 2-iodo-aromatic ether is

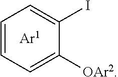

The above technical solution can be expressed as follows:

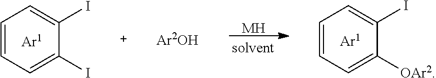

In the present invention, Ar¹ is benzene, naphthalene, and the like; Ar² is benzene, naphthalene, pyridine, quinoline, pyrimidine, thiophene, and the like; M is sodium, potassium, and lithium.

### Beneficial Effects

The preparation method of the 2-iodo-aromatic ether of the present invention has the following advantages:
1) transition metal catalysts are not needed to be added in the coupling process, so that metal pollution to products is avoided;
2) the method can be carried out at room temperature, it has high functional group compatibility, and it solves the problem that the coupling reaction of the existing metal catalysis into the aromatic ether needs to be carried out at higher temperature;
3) the reaction is simple, the reagent is cheap, and the required cost is low;
4) it can prepare the products that are not easily prepared by other methods, such as products with iodine in both aromatic rings.

### BRIEF DESCRIPTION OF THE DRAWINGS

FIG. 1 shows the hydrogen nuclear magnetic resonance spectrum of the product of 2-iodo-aromatic ether 3aa.

### EXAMPLES OF THE PRESENT INVENTION

The coupling reaction of phenol and iodide does not need any catalyst, the operation is simple, the by-product of the reaction is only sodium iodide, and no toxic substances are generated; the 2-iodo-aromatic ether prepared by the present invention can be obtained by only one-step mild reaction without excessive coupling products, and is superior to all existing methods.

The technical scheme of the invention is as follows: An alkali metal hydride was suspended in a solvent, the phenol was added, and then 1, 2-iodo-aromatic hydrocarbon was added for reaction for 0.2-10 hours at room temperature. After that, water was added to terminate the reaction, and the solvent was extracted and evaporated to dry for column chromatography purification to obtain the product of 2-iodo-aromatic ether.

The raw materials involved in the present invention are all commercially available products; the specific preparation and testing methods used are existing techniques; the reaction of the present invention does not require inert gas, and materials are added to the reaction bottle in a conventional environment. After the addition is completed, cover the bottle for reaction; the specific operation and purification are conventional techniques.

### Example 1

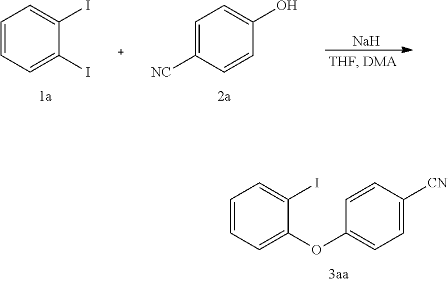

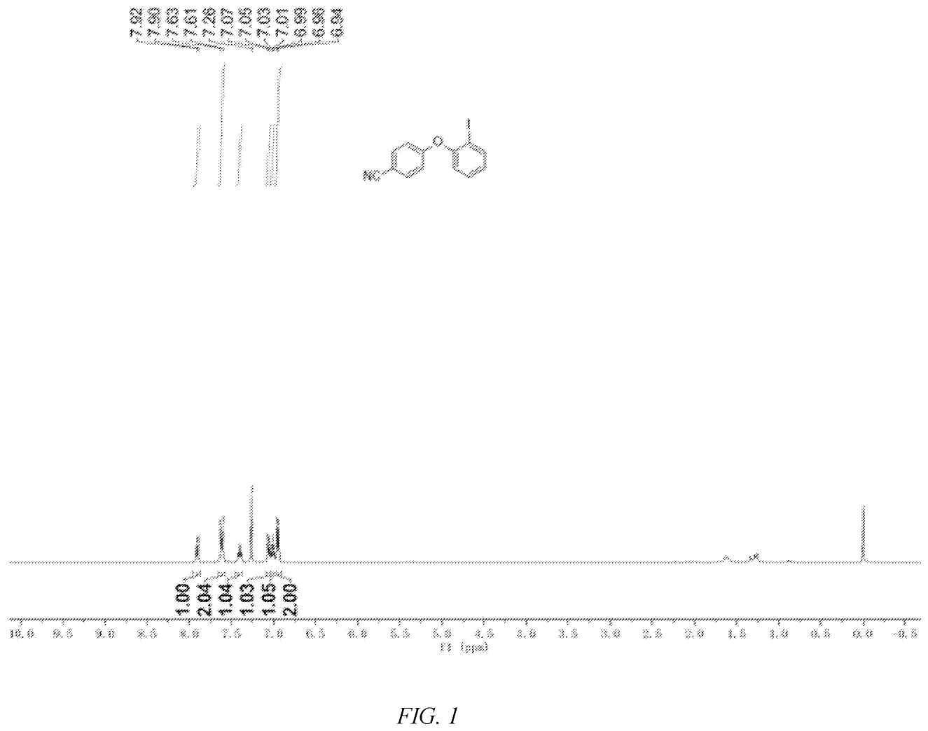

Sodium hydride (60% in oil, 36 mg, 0.9 mmol, 3 eq.) was suspended in THF (1 mL), and it was conventionally stirred and added into a solution of phenol 2a (36 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 2 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3aa with the yield of 99%. The hydrogen nuclear magnetic resonance spectrum is shown in FIG. 1, LR-MS (ESI): m/z 322.1 [M+H]⁺.

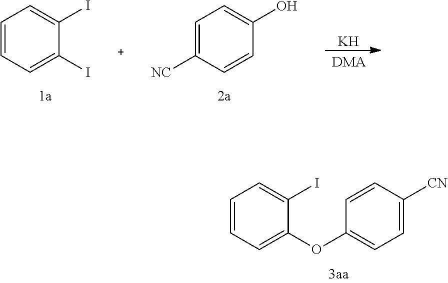

The aforementioned sodium hydride was replaced with an equal molar amount of potassium hydride, with the rest keeping unchanged, to obtain the product of iodo-aromatic ether 3aa with a yield of 39%.

The aforementioned sodium hydride was replaced with an equal molar amount of lithium hydride, with the rest keeping unchanged, and it could not obtain the product of iodo-aromatic ether 3aa.

The aforementioned sodium hydride was replaced with an equal molar amount of calcium hydride, with the rest keeping unchanged, and it could not obtain the product of iodo-aromatic ether 3aa.

The aforementioned room temperature was replaced with ice water bath, with the rest keeping unchanged, to obtain the product of iodo-aromatic ether 3aa with a yield of 51%.

The amount of the aforementioned sodium hydride was changed to be 2 eq, with the rest keeping unchanged, to obtain the product of iodo-aromatic ether 3aa with a yield of 81%.

The aforementioned reaction for 2 hours at room temperature was replaced with the reaction for 1 hour at room temperature, with the rest keeping unchanged, to obtain the product of iodo-aromatic ether 3aa with a yield of 96%.

### Example 2

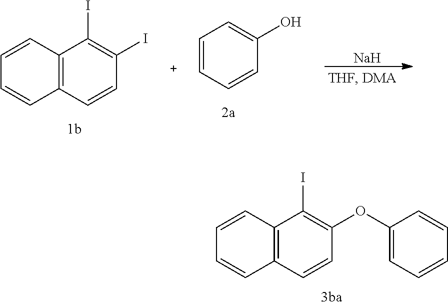

Sodium hydride (60% in oil, 36 mg, 0.9 mmol, 3 eq.) was suspended in THF (1 mL), and it was conventionally stirred and added into a solution of phenol 2a (28 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ba with the yield of 35%.

### Example 3

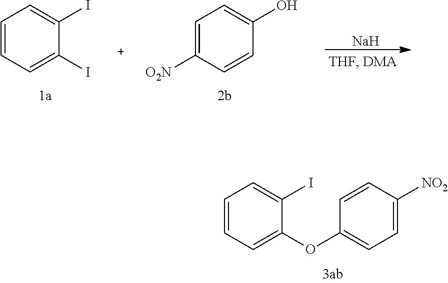

Sodium hydride (60% in oil, 36 mg, 0.9 mmol, 3 eq.) was suspended in THF (1 mL), and it was conventionally stirred and added into a solution of phenol 2b (42 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 0.5 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ab with the yield of 97%.

¹H NMR (400 MHZ, CDCl₃) δ: 8.22 (d, J=7.8 Hz, 2H), 7.92 (d, J=7.5 Hz, 1H), 7.43 (t, J=7.5 Hz, 1H), 7.09 (d, J=7.5 Hz, 1H), 7.03 (t, J=7.2 Hz, 1H), 6.96 (d, J=7.8 Hz, 2H). LR-MS (ESI): m/z 342.1 [M+H]⁺.

### Example 4

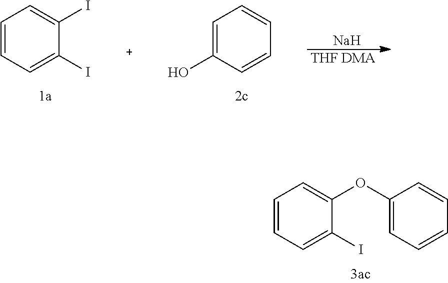

Sodium hydride (60% in oil, 36 mg, 0.9 mmol, 3 eq.) was suspended in THF (1 mL), and it was conventionally stirred and added into a solution of phenol 2c (28 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 0.5 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ac with the yield of 98%.

¹H NMR (400 MHZ, CDCl₃) δ 7.87 (dd, J=7.8, 1.5 Hz, 1H), 7.42-7.32 (m, 2H), 7.32-7.26 (m, 1H), 7.18-7.07 (m, 1H), 7.02-6.94 (m, 2H), 6.94-6.83 (m, 2H). LR-MS (ESI): m/z 297.1 [M+H]⁺.

### Example 5

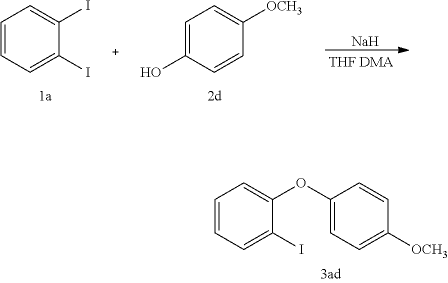

Sodium hydride (60% in oil, 36 mg, 0.9 mmol, 3 eq.) was suspended in THF (1 mL), and it was conventionally stirred and added into a solution of phenol 2d (37 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ad with the yield of 89%.

¹H NMR (400 MHZ, CDCl₃) δ:7.84 (dd, J=7.8, 1.5 Hz, 1H), 7.24 (ddd, J=8.8, 5.7, 1.5 Hz, 1H), 7.02-6.92 (m, 2H), 6.92-6.86 (m, 2H), 6.81 (td, J=7.7, 1.4 Hz, 1H), 6.76 (dd, J=8.2, 1.3 Hz, 1H), 3.81 (s, 3H). LR-MS (ESI): m/z 327.2 [M+H]⁺.

### Example 6

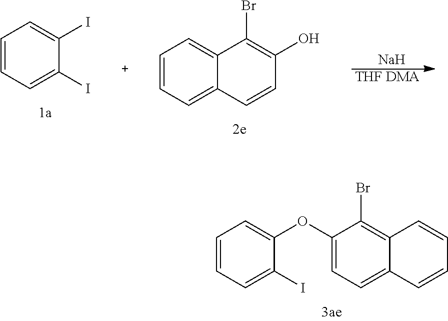

Sodium hydride (60% in oil, 36 mg, 0.9 mmol, 3 eq.) was suspended in THF (1 mL), and it was conventionally stirred and added into a solution of phenol 2e (38 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ae with the yield of 91%.

¹H NMR (400 MHZ, CDCl₃) δ 8.31 (d, J=8.5 Hz, 1H), 7.89 (t, J=7.1 Hz, 1H), 7.82 (dd, J=17.9, 8.5 Hz, 2H), 7.64 (t, J=7.6 Hz, 1H), 7.52 (t, J=7.4 Hz, 1H), 7.23 (d, J=8.1 Hz, 1H), 7.11 (d, J=8.7 Hz, 1H), 6.87 (t, J=7.4 Hz, 1H), 6.71 (d, J=8.1 Hz, 1H). LR-MS (ESI): m/z 424.7 [M+H]⁺.

### Example 7

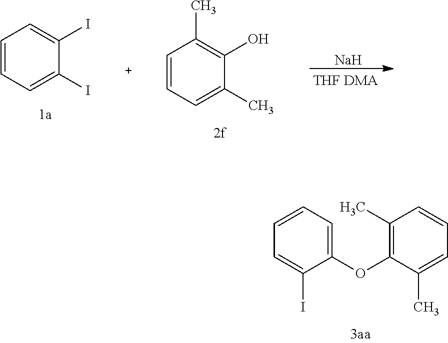

Sodium hydride (60% in oil, 36 mg, 0.9 mmol, 3 eq.) was suspended in THF (1 mL), and it was conventionally stirred and added into a solution of phenol 2f (34 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3af with the yield of 85%.

¹H NMR (400 MHZ, CDCl₃) δ:7.85 (d, J=7.6 Hz, 1H), 7.19-7.04 (m, 4H), 6.74 (dd, J=15.2, 7.7 Hz, 1H), 6.30 (d, J=8.1 Hz, 1H), 2.13 (s, 6H). LR-MS (ESI): m/z 325.1 [M+H]⁺.

### Example 8

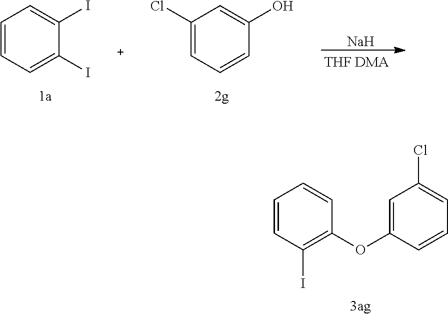

Sodium hydride (60% in oil, 36 mg, 0.9 mmol, 3 eq.) was suspended in THF (1 mL), and it was conventionally stirred and added into a solution of phenol 2g (38 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 0.5 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ag with the yield of 96%.

¹H NMR (400 MHZ, CDCl₃) δ: 7.88 (dd, J=7.9, 1.5 Hz, 1H), 7.36-7.30 (m, 1H), 7.26 (dd, J=9.2, 7.1 Hz, 1H), 7.08 (ddd, J=8.0, 1.9, 0.8 Hz, 1H), 7.01-6.89 (m, 3H), 6.85 (ddd, J=8.3, 2.4, 0.8 Hz, 1H). LR-MS (ESI): m/z 331.0 [M+H]⁺.

### Example 9

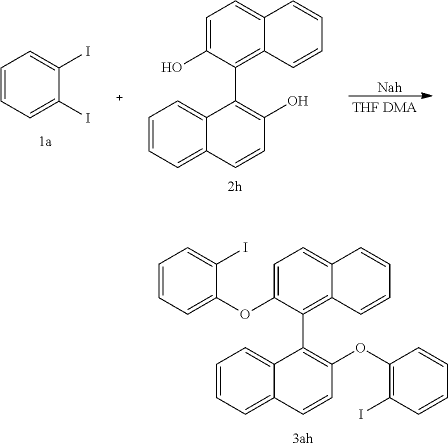

Sodium hydride (60% in oil, 72 mg, 1.8 mmol, 6 eq.) was suspended in THF (1 mL), and it was conventionally stirred and added into a solution of phenol 2h (86 mg, 0.3 mmol, 1 eq.) in DMA (0.4 mL), and then it was stirred for 10 minutes at room temperature. After that, it was added to a solution of 1a (297 mg, 0.9 mmol, 3 eq.) in THF (0.6 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3ah with the yield of 89%.

¹H NMR (400 MHZ, CDCl₃) δ 7.90 (t, J=7.4 Hz, 4H), 7.66 (d, J=7.7 Hz, 2H), 7.47 (d, J=8.4 Hz, 2H), 7.41 (dd, J=15.2, 7.6 Hz, 2H), 7.33 (t, J=7.5 Hz, 2H), 7.13 (d, J=9.0 Hz, 2H), 7.04 (t, J=7.6 Hz, 2H), 6.70 (t, J=6.9 Hz, 4H). LR-MS (ESI): m/z 690.8 [M+H]⁺.

### Example 10

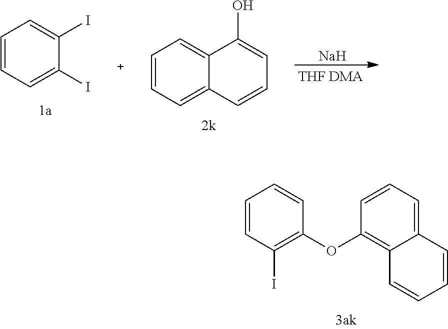

Sodium hydride (60% in oil, 36 mg, 0.9 mmol, 3 eq.) was suspended in THF (1 mL), and it was conventionally stirred and added into a solution of phenol 2k (43 mg, 0.3 mmol, 1 eq.) in DMA (0.3 mL), and then it was stirred for 10 minutes at room temperature. After that, it was added to a solution of 1a (148 mg, 0.45 mmol, 1.5 eq.) in THF (0.2 mL) for reaction for 1 hours at room temperature. Then water was added for quenching and it was extracted with ethyl acetate three times and the organic layers were combined and dried with sodium sulfate, evaporated to dryness and purified by column chromatography to obtain the product of iodo-aromatic ether 3aj with the yield of 85%.

¹H NMR (400 MHZ, CDCl₃) δ 8.25-8.16 (m, 1H), 7.89 (ddd, J=9.4, 7.4, 2.6 Hz, 2H), 7.63 (d, J-8.3 Hz, 1H), 7.58-7.45 (m, 2H), 7.41-7.32 (m, 1H), 7.28-7.20 (m, 1H), 6.91-6.76 (m, 3H). LR-MS (ESI): m/z 347.2 [M+H]⁺.

The products of the Examples of the present invention matched with the design through the nuclear magnetic resonance testing and mass spectrometry testing. Compared with the prior art, the present invention can obtain the 2-iodo-aromatic ether only by stirring commercial 1, 2-iodo-aromatic hydrocarbon and phenol for a period of time at room temperature under the action of alkali metal hydride, and is obviously superior to the existing methods in terms of synthesis efficiency and cost.

1. A method for preparing a 2-iodo-aromatic ether under the action of alkali metal hydrides characterized in that it comprises the following steps: reacting a 1, 2-iodo-aromatic hydrocarbon with a phenol in the presence of an alkali metal hydride to obtain the 2-iodo-aromatic ether.

2. The method for preparing a 2-iodo-aromatic ether under the action of alkali metal hydrides according to claim 1, wherein the alkali metal hydride and the phenol are added into a solvent, and then the 1, 2-iodo-aromatic hydrocarbon is added to obtain the 2-iodo-aromatic ether.

3. The method for preparing a 2-iodo-aromatic ether under the action of alkali metal hydrides according to claim 2, wherein the solvent is one or more selected from the group consisting of DMF, DMA, THF, 2-MeTHF, DME, MTBE, diethyl ether, DMSO, NMP, and toluene.

4. The method for preparing a 2-iodo-aromatic ether under the action of alkali metal hydrides according to claim 1, wherein the alkali metal hydride is sodium hydride, potassium hydride or lithium hydride

5. The method for preparing a 2-iodo-aromatic ether under the action of alkali metal hydrides according to claim 1, wherein the phenol is phenol, substituted phenol or heterocyclic phenol.

6. The method for preparing a 2-iodo-aromatic ether under the action of alkali metal hydrides according to claim 1, wherein a mole ratio of the phenol to the 1,2-iodo-aromatic hydrocarbon to the alkali metal hydride is 1:(0.1-10):(1.2-10).

7. The method for preparing a 2-iodo-aromatic ether under the action of alkali metal hydrides according to claim 1, wherein a reaction temperature is 0-100° C.; a reaction time is 0.2-10 hours.

8. 2-Iodo-aromatic ether prepared by the method for preparing 2-iodo-aromatic ether under the action of alkali metal hydrides according to claim 1.

9. An application of an alkali metal hydrides, with a 1, 2-iodo-aromatic hydrocarbon and a phenols as starting materials, in preparing a 2-iodo-aromatic ether.

10. The application according to claim 9, wherein the chemical structural formula of the 1, 2-iodo-aromatic hydrocarbon is: 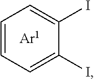
wherein Ar¹ is benzene or naphthalene.
